(19) United States
(12) Patent Application Publication
(10) Pub. No.: US 2016/0128935 A1
(43) Pub. Date: May 12, 2016

(54) METHOD OF TREATING AN ANIMAL

(71) Applicants: Kyle Clark Howes, Herriman, UT (US); Jeremy James Olsen, Sandy, UT (US)

(72) Inventors: Kyle Clark Howes, Herriman, UT (US); Jeremy James Olsen, Sandy, UT (US)

(21) Appl. No.: 14/988,434

(22) Filed: Jan. 5, 2016

Related U.S. Application Data
(63) Continuation of application No. 13/850,765, filed on Mar. 26, 2013, now abandoned.
(60) Provisional application No. 61/615,750, filed on Mar. 26, 2012.

(51) Int. Cl. A61K 9/00; A61K 31/122; A61K 31/4164; A01K 61/00; A01K 99/00

(52) U.S. Cl. CPC A61K 9/0056; A23K 1/17; A23K 1/188; A23K 1/1606; A01K 61/00; A01K 99/00; A61K 31/4164; A61K 31/122

(57) ABSTRACT

There is a method of treating an animal including the step of providing a live food creature configured to be consumable to an animal to be treated. The method includes applying a beneficial substance to the live food creature such that when consumed, the beneficial substance is consumed along with the live food creature. The step of applying a beneficial substance includes injecting the beneficial substance into the live food creature. The method may include the step of feeding the live food creature to the animal to be treated.

### CROSS-REFERENCE TO RELATED APPLICATIONS

This application is a Continuation application of, under 35 U.S.C. §121, and claims priority to, under 35 U.S.C. §121, United States Non-Provisional application Ser. No. 13/850,765, entitled A Method of Treating an Animal, by Kyle Clark Howes et al., filed on Mar. 26, 2013. This invention also claims priority, under 35 U.S.C. §120, to the U.S. Provisional Patent Application No. 61/615,750 to Kyle Clark Howes et al. filed on Mar. 26, 2012, which is incorporated by reference herein.

### BACKGROUND OF THE INVENTION

1. Field of the Invention

The present invention relates to medication delivery methods and systems, specifically to a method of treating an animal.

2. Description of the Related Art

Veterinary medicine is the branch of science that deals with the prevention, diagnosis and treatment of disease, disorder and injury in animals. The scope of veterinary medicine is wide, covering all animal species, both domesticated and wild, with a wide range of conditions which can affect different species. It includes diagnosing and treating a wide variety of maladies in a wide variety of animals. Treatments sometimes include providing the animal(s) with medications. However, there is some difficulty in medicating animals. In particular, one cannot simply inform the animal that they should take their medicine and expect the animal to take it. Animals tend to be resistant and/or reluctant to take medication. Further, biological differences between animals create additional barriers to proper treatment and adequate dosing. Still more, some animals, such as fish, share a feeding/breathing/living medium that if dosed, requires dosing all of the animals in the space in order to ensure that a particular animal is dosed. When this occurs, much more medicine is needed and the additional animals are unnecessarily exposed to medicinal side effects.

Veterinary medicine is widely practiced, both with and without professional supervision. Professional care is most often led by a veterinary physician (also known as a vet, veterinary surgeon or veterinarian), but also by para-veterinary workers such as veterinary nurses or technicians. This can be augmented by other paraprofessionals with specific specialties such as animal physiotherapy or dentistry, and species relevant roles such as farriers. Often owners and/or caretakers of animals will be required to step up and handle health issues of animals, including but not limited to properly dosing the animals with the appropriate medications. Such individuals often do not have the training, expertise, and experience to administer to all the needs of an animal and may make mistakes in amounts, timing, and etc.

Veterinary science helps human health through the monitoring and control of zoonotic disease (infectious disease transmitted from non-human animals to humans) and veterinary scientists often collaborate with epidemiologists. However, it is typically hard to treat and medicate certain types of animals depending on their disease and their environment.

Some improvements have been made in the field. Examples of references related to the present invention are described below in their own words, and the supporting teachings of each reference are incorporated by reference herein:

U.S. Pat. No. 4,129,955, issued to Schommer, discloses an article in the shape of a handgun for injecting air into live bait, the device including a generally transparent barrel member having hypodermic needle centrally disposed with respect to the barrel and recessed therein. The barrel is stationary and coacts with a movable spring loaded plunger having a check valve in the closed end thereof, the plunger being actuable by the trigger of the gun to pass air through a spring loaded check valve into live bait such as a worm or the like.

U.S. Pat. No. 5,725,892, issued to Gibbs, discloses a mold utilized for creating a fishing worm having an abdominal cavity that contains an attractant gel having a stimulating taste to a fish, the mold having first and second sections, with each section having a cavity portion. Each cavity portion of the mold represents a part of a desired worm configuration, with the cavity portion of one mold section being able to be brought into careful alignment with the cavity portion of the other mold section, after which molten plastic can be injected into the completed mold cavity constituted by the aligned cavity portions of the first and second mold sections. An elongate plug held in place by a small diameter supporting member is mounted in a mid portion of the cavity of the first mold section, at a position corresponding to the abdominal region of the worm to be created. Thus, upon molten plastic being inserted into the completed mold cavity, a plastic worm will be defined, with such worm having an abdominal cavity resulting from the utilization of the elongate plug. The small diameter supporting member causes a hole to be made in the sidewall of each plastic worm so created, through which the attractant gel having a pleasant taste to a fish can thereafter be injected.

U.S. Pat. No. 6,240,876, issued to Giannaris, discloses a method for coloring live bait worms comprises adding to a cultivation bed containing one or more worm storage media, a non-toxic amount of a colorant to form a coloring medium, and incubating live bait worms in the coloring medium at a temperature and for a time sufficient to allow the bait worms to become colored by ingesting the coloring medium. The colorant is preferably a D&C coloring additive such as a xanthene dye, most preferably D&C Yellow No. 8, and/or D&C Yellow No. 7, and the worm storage medium is preferably peat loam. The worms become colored during an incubation period of up to about one week and retain their color for a prolonged period of time after being removed from the coloring medium.

U.S. Patent Application Publication No.: 2008/0003326, by Olive et al., discloses a method of improving the nutritional content of marine worms, such as polychaetes, by feeding the worms a diet having a concentration of pigments, polyunsaturated fatty acids, lipids, vitamins and/or minerals sufficient to enhance the level of such components within the tissue of the worms. The worms can then be used for aquaculture, for example in farming marine fish and/or shrimps. One component of particular benefit is astaxanthin, which is preferably present in the polychaete diet of the worms at a concentration of at least 200 ppm. Advantageously the diet fed to the worms will include at least 10% by weight of vegetate oil. Conveniently the worms may be dried by lyophilisation or by refractance window drying before optionally being included or formed into pellets for aquaculture or other (i.e. aquarium) use.

The inventions heretofore known suffer from a number of disadvantages which include but are not limited to failing to adequately measure dosing, not being palatable to the animal, having a low absorption rate, having low compliance with medication schedules, not being natural, being expensive, wasting medicine, causing trauma and/or confusion to treated animals, treating non-sick animals, not being targeted, failing to treat animals that are too sick to desire to eat without instinctual feeding impulses, failing to treat finicky eaters, requiring too much handling of animals, being difficult to apply, not being entertaining, allowing for too many dosing mistakes, being limited in the kinds of treatments and/or treatment protocols available, being inconsistent with natural animal tendencies/instincts, causing stress to animals, not allowing for centralized controls, having a high likelihood of accidental dosing of humans (especially children), being dangerous, causing side effects to handlers through exposure during dosing, and/or causing side effects to other animals.

What is needed is a method of treating animals that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available methods of treating animals. Accordingly, the present invention has been developed to provide a safe and efficient method of treating animals.

According to one embodiment of the invention, there is a method of treating an animal. The method may include the step of providing a live food creature that may be configured to be consumable to an animal to be treated. The live food creature may be selected from the group of live food creatures consisting of worms, crickets, fish, and shrimp and wherein the animal to be treated is selected from the group of animals consisting of fish; frogs, reptiles, and spiders.

The method may include applying a beneficial substance to the live food creature such that when consumed, the beneficial substance may be consumed along with the live food creature. The beneficial substance may be selected from the group of beneficial substances consisting of: vitamin supplements, color-enhancers, medicine, anti-fungal agents, antibiotics, probiotics, and antioxidants. The step of applying a beneficial substance may consist essentially of injecting the beneficial substance into the live food creature. The step of applying may consist essentially of causing the live food creature to ingest the beneficial substance. The step of applying may consist essentially of causing the beneficial substance to be absorbed into the live food creature transdermally.

The method may include the step of feeding the live food creature to the animal to be treated. The method may include the step of causing the live food creature to have a lower metabolic rate, thereby reducing the rate at which an effective dosage level of the beneficial substance is reduced. The step of causing the live food creature to have a lower metabolic rate may be selected from the group of rate lowering methods consisting of temperature reduction, light treatment, and chemically induced hibernation.

The method of treating an animal may include the step of marking the live food creature according to a protocol configured to identify the live food creature as having a particular characteristic associated with the step of applying a beneficial substance to the live food creature. The particular characteristic may be selected from the group of characteristics consisting of beneficial substance name, application date, metabolic rate, shelf life, and dose level. The step of marking may be performed according to a marking technique selected from the group of marking techniques consisting of dying, tattooing, etching, and cutting.

The method of treating animals may include the step of shipping the live food creature to a remote location where the step of feeding is to occur. The method may include applying a second beneficial substance to the live food animal after the step of shipping.

According to one embodiment of the invention, there is a live food creature that may include a beneficial substance not natural to the live food creature.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a conceptual view of a method of treating an animal, according to one embodiment of the invention;

FIG. 2 is a perspective view of a method of applying a medicine to a live food creature in preparation for treating an animal, according to one embodiment of the invention;

FIG. 3 is a flow chart of a method of treating an animal, according to one embodiment of the invention; and

FIG. 4 is a flow chart of method of treating an animal, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

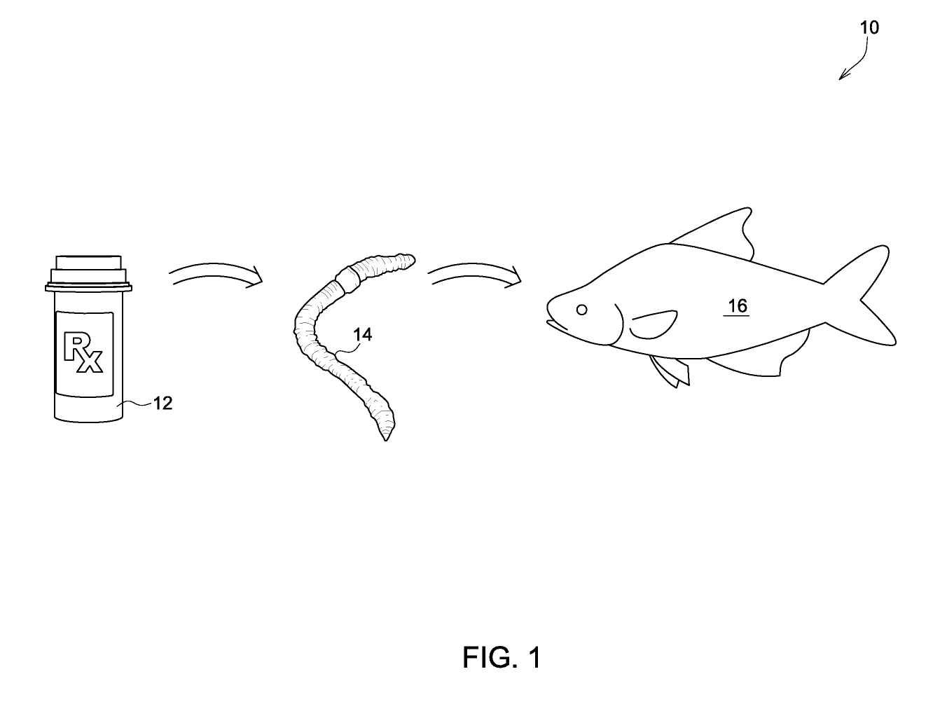

FIG. 1 is a conceptual view of a method of treating an animal, according to one embodiment of the invention. There is shown a method of treating an animal 10 including medication 12 being applied to a live creature 14, which is fed to an animal 16.

The illustrated method of treating an animal 10 includes taking live bait type creatures 14 (worms, maggots, bugs, etc.) and using them as a carrier for medical treatment, color enhancement, parasite control, etc. The treatment is injected or applied to the live creature 14, thereby introducing medicine, supplements, enhancements, etc. into the live food creature 14 and then using the live food creature 14 as feed for the animal 16 or animals that are intended to be treated. In particular, the method includes taking a live food creature 14 and including the supplement or medicine 12 within the body of the live food creature 14, thereby using the live food creature 14 as a vehicle for delivery to a target animal 16.

The illustrated method of treating an animal 10 includes using live creatures 14 such as worms, maggots, and bugs to be used as the catalyst for medical treatment, color enhancement, and parasite control in fish, ornamental fish, reptiles, poultry, and birds. The catalyst or medicine 12 is fed to the fish or reptile after being treated, fed, grown in, injected, absorbed, or sprayed with such applicable chemical or additives such as antibiotics, vitamins, astaxanthins, herbs, and natural oils for the treatment and/or enhancement of certain ailments and conditions of the fish or reptile.

The following are a list of non-limiting examples of chemicals or additives used in the method of treating an animal: kanamyclin (antibiotic), tetracycline (antibiotic), erythromycin (antibiotic), astaxanthin (color enhancer), magnesium sulfate (Epson salt, parasite killer), metronidazole (internal parasite killer), and minocycline (antibiotic). If one included the aforementioned list of chemicals or additives into feed for fish, 90% of most fish issues would be taken care of. Epson salt effectiveness on treating internal parasites in fish may also be used. Maggots, in addition to worms, grow fast and retain most of what they eat, thereby providing an effective vehicle to deliver the chemicals or additives to the targeted animal. After the chemicals or additives are injected or applied to the live creature bait 14, the live creature bait 14 may be freeze dried, frozen, or kept live.

The illustrated live food creature 14 may include a beneficial substance (foreign substance, desired substance, etc.) 12 not natural to the live food creature 14. Accordingly, the live food creature is not found in nature to have that substance therein and/or contain the substance in such amounts/concentrations.

In one non-limiting example, there is a method of applying a desired substance (foreign substance) to live food creatures and then feeding those live creatures to an animal for a specific and dosed effect on that particular animal. The applied substance will be a substance not otherwise found in the live food creature and/or not otherwise found in the live food creature in the amount and/or concentration that occurs when the foreign substance is so added.

Applying may be done by various ways, including but not limited to injection, absorption, ingestion (especially where the substance is not something that the animal would ingest but the live food creature may naturally ingest or may be coerced into ingesting), etc. The application process should not immediately kill the live food creature, as the live creature is more likely to be eaten by the intended animal. The application process should also not substantially change the characteristics of the live food creature that are important to the animal properly recognizing the live food creature as being desirable food. Accordingly, methods that dramatically change the shape, size, texture, color and etc. (where relevant to such identification) should be avoided.

The desired substance may be a medicine, food, vitamin, supplement, and etc. There may be a plurality of desired substances applied simultaneously and/or consecutively and/or at varying points in the process. The desired substance may be

The live creature may be processed to reduce its metabolism so that the applied substance is not metabolized, but the creature is still alive and can be shipped. The processing to reduce metabolic rate may be by various ways, including but not limited to by temperature reduction, application of light frequencies, application of metabolic reducers that do not cause problems for the animal when ingested. It may be that the method of reducing metabolic rate triggers a state of hibernation in the live creature.

The live creature (and/or its packaging) may be marked to indicate the substance, date, and/or the dose. Marking may be by various ways, including but not limited to by use of dyes, etching, attaching markers, and/or may be delivered simultaneously with the substance so that there is correlation between application of the substance and the marking, and etc. Marking may be applied using marking tools, colors, devices, etc. that are not detectable by the animal intended to be treated. As a non-limiting example, such marking may be applied to a region of the live food creature not seen when feeding. As another non-limiting example, a dye may be used that generates a color that is not in the visible spectrum of the animal to be treated. One may apply the substance to the creature prior to and/or after shipping the creature to the end user.

The live food creatures may be worms (mealworms, bloodworms, blackworms, nightcrawlers, maggots, etc.), crickets, fish, brine shrimp, and etc. Live food creatures will be generally smaller than the animals they are intended to feed. Further, live food creatures are generally kept in a whole state (not cut, ground, chopped, etc.) so that their live state persists through the step of feeding the live food creature to the animal. It is beneficial if the live food creature is able to move so that it can incite instinctual feeding behaviors in the animal. Accordingly, it is helpful if the step of applying the foreign substance to the live food creature is done in a manner that does not immediately kill the live food creature. Similarly, the foreign substance should be a substance and in an amount that does not immediately kill the live food creature.

Animals to be treated may be fish (decorative, farm, etc.), reptiles, frogs, birds, spiders, toads, and etc. Generally, the live food creature is selected based on the intended animal to be treated. For example, in the case of reptile and frogs, the live food creature may be best selected among the list of insects (bugs).

Advantageously, such a system facilitates the proper and timely dosing of animals with appropriate amounts of medicines (and/or etc.) in a manner that is easier and safer for the animals and their handlers. Further, applicant has experienced a large increase in dosing protocol compliance with animals, especially with fish, wherein the animals are much more likely to receive the intended dose, even when their appetites are suppressed due to sickness. Accordingly, these animals are getting better faster and in some cases animals lives are saved that would otherwise have been lost. Further, less time is spent by the handler in keeping the animals on their dosing schedule. Wherein some decorative fish are extremely valuable ($1,000-$100,000+) and wherein commercial animal handling operations are run on ever slimming margins, it is more and more critical to have greater success in keeping the animals healthy.

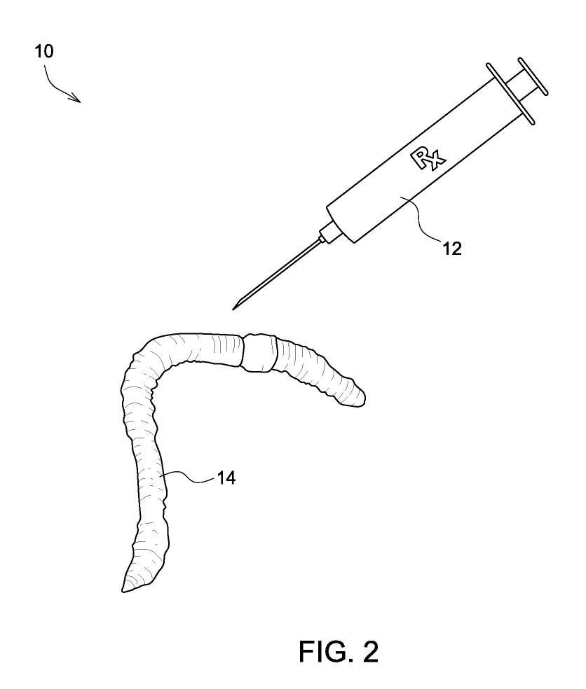

FIG. 2 is a perspective view of a method of applying a medicine to a live food creature in preparation for treating an animal, according to one embodiment of the invention. There is shown a method of treating an animal 10 includes injecting a live creature 14 with medicine or supplements 12.

The illustrated method of treating an animal 10 includes injecting medicine or supplements for medical treatment, color enhancement, parasite control, etc. into a live creature 14. The medicine or supplements 12 are injected or applied to the live creature 14, thereby introducing medicine, supplements, etc. into the live food creature 14 and then using the live food creature 14 as feed for the animals intended to be treated. In particular, the method includes taking a bait creature 14 and including the supplement/medicine 12 within the body of the bait creature 14 and using that as a vehicle for delivery to a target animal.

The method of treating an animal 10 is configured to include a live food creature 14, such as: any kind of suitable worm (red wiggler, nightcrawler, black worms, blood worms, etc.), maggots, bugs (flies, crickets, grasshoppers, cockroaches, etc.), and etc. The medicines/supplements/additives 12 being applied or injected may be: antibiotics, parasite treatments, color enhancements, vitamins, nutrients, proteins, herbs, etc. The method of treating an animal 10 includes delivery methods, such as: injection, absorption through skin, ingestion, sprayed, coated, etc.

The following list is a non-limiting set of exemplary medicines and etc. that may be applied to a live food creature:
ANTIBIOTICS
   Tetracycline
   Kanamycin
   Erythromycin
   Minocycline
   Metronidazole
   Amoxicylin
NATURAL FOOD COLOR ENHANCERS, VITAMINS, ANTIOXIDANTS & VITAMIN FOR IMMUNE BOLSTERING
   Astaxanthin
   Magnesium Sulfate
   Cyclop-eeze (type of small shrimp)
   Krill
   Daphnia
   Brine Shrimp
   Spirulina
   Amino Acids
   Vitamins A+ D+ and E, B12
   Vitamins A, C, D3, Niacin, Folic Acid Garlic as a Natural antioxidant/antibiotic
PROBIOTICS
   Lactic acid bacteria
      Lactobacillus
      Carnobacterium
   Vibrio genus bacteria (e.g. V. Alginolyticus)
   Bacillus genus bacteria
   Pseudomonas genus bacteria
   Aeromonas
   Flavobacterium

### Treatment Example 1

Discus fish having parasites were treated using one live food creature (a live worm) per fish for three days. Each worm was injected with a name brand parasite killer at a measurement of 5 ml in syringe and the worm was injected with as much of the 5 ml as the worm would hold. The injected worms were fed to the fish individually by placing the worm adjacent the fish and feeding was confirmed by observation. The fish ate the treated worms in all cases, showing a vigorous feeding instinct for the live worms. The treated fish were sufficiently recovered to be salable within four days, which is a faster recovery time than is generally experienced with traditional treatment methods.

### Treatment Example 2

Emperor Cichlids and Peacock Cichlids (fish) having bacterial infections were treated using one live food creature (a live worm) per fish for six days. Each worm was injected with Antibiotic Metronidazole at a measurement of 5 ml in syringe and the worm was injected with as much of the 5 ml as the worm would hold. The injected worms were fed to the fish individually by placing the worm adjacent the fish and feeding was confirmed by observation. The fish ate the treated worms in all cases, showing a vigorous feeding instinct for the live worms. The treated fish were sufficiently recovered to be salable within seven days, which is a faster recovery time than is generally experienced with traditional treatment methods. It is typical to lose some fish being treated for bacterial infections. None of the fish so treated were lost.

### Treatment Example 3

A quantity of live food creatures (worms) were present in a worm bed wherein a quantity of Astaxanthin had been placed. The worms ingested the Astaxanthin almost entirely, thereby incorporating the same within their bodies in preparation for being fed to fish, and thereby providing enhanced coloration and health to the same.

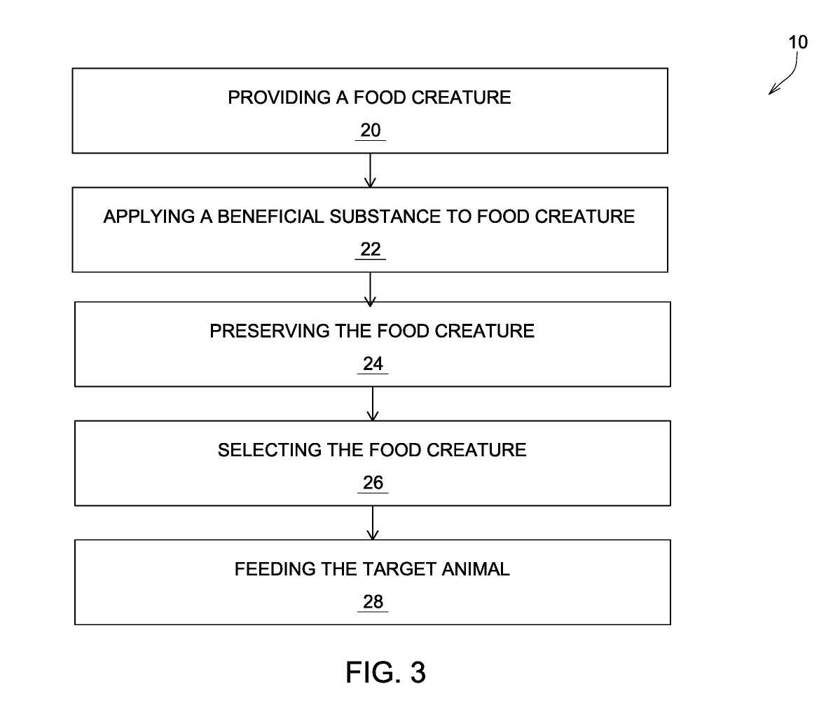

FIG. 3 is a flow chart of a method of treating an animal, according to one embodiment of the invention. There is shown a method of treating an animal 10 including the steps of providing a food creature 20, applying a beneficial substance to the food creature 22, preserving the food creature 24, selecting the food creature 26, and feeding the target animal 28

The illustrated method of treating an animal 10 includes the step of providing a live food creature that is configured to be consumable to an animal to be treated 20. The live food creature is selected from the group of live food creatures consisting of worms, crickets, fish, and shrimp and wherein the animal to be treated is selected from the group of animals consisting of fish; frogs, reptiles, and spiders.

The method 10 includes applying a beneficial substance to the live food creature 22 such that when consumed, the beneficial substance is consumed along with the live food creature. The beneficial substance is selected from the group of beneficial substances consisting of: vitamin supplements, color-enhancers, medicine, anti-fungal agents, antibiotics, probiotics, and antioxidants. The step of applying a beneficial substance may consist essentially of injecting the beneficial substance into the live food creature. The step of applying 22 consists essentially of causing the live food creature to ingest the beneficial substance. The step of applying 22 consists essentially of causing the beneficial substance to be absorbed into the live food creature transdermally. The step of applying a beneficial substance to the live food creature may include the step of changing a color of the bait by using additives.

The method of treating an animal 10 includes the step of preserving the food creature 24. Preserving the food creature 24 includes a stabilization process for shipping/storage/etc. of live creatures being freeze dried, heated, frozen, paralyze, oil-base treatment, etc. The method of treating an animal 10 includes selecting the food creature to be fed to a particular animal with a particular need or illness 26. The method includes feeding the target animal 28, according to dosing techniques, such as rating the bait (numbers, colored packaging, colored additives to the bait, etc.) to give dosing information; thereby associating that with size/weight of fish or animal to know how much to feed the fish or animal. The dose of the live creature bait is according to a protocol/portfolio of products that aligns specific bait sizes, colors, weights, types, etc. with particular dosages of particular additives and use that protocol to enhance the safe delivery and dosing of the fish or animal by making it easier and more fool-proof for users to know what the dose should be, in addition, to knowing the contents of the live creature bait. Marking indicators on the live creature bait makes overdosing and/or other undesired interactions impossible according to fish or animal type/size and/or additive type.

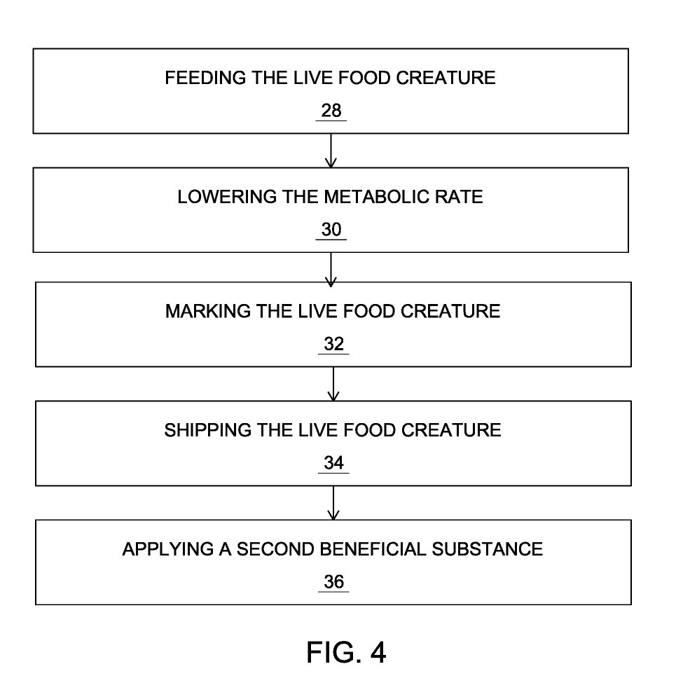

FIG. 4 is a flow chart of method of treating an animal, according to one embodiment of the invention. There is shown a method of treating an animal 10 including the steps of lowering the metabolic rate of the live creature bait 30, marking the live creature bait 32, and shipping the live creature bait 34.

The illustrated method of treating an animal 10 includes the step of feeding the live food creature to the animal to be treated 28. Feeding the live food creature to the animal causes the creature to have a lower metabolic rate 30, thereby reducing the rate at which an effective dosage level of the beneficial substance is reduced. The step of causing the live food creature to have a lower metabolic rate 30 is selected from the group of rate lowering methods consisting of temperature reduction, light treatment, and chemically induced hibernation.

The method of treating an animal 10 includes the step of marking the live food creature 32 according to a protocol configured to identify the live food creature as having a particular characteristic associated with the step of applying a beneficial substance to the live food creature. The particular characteristic is selected from the group of characteristics consisting of beneficial substance name, application date, metabolic rate, shelf life, and dose level. The step of marking is performed according to a marking technique selected from the group of marking techniques consisting of dying, tattooing, etching, and cutting.

The method of treating animals 10 includes the step of shipping the live food creature 34 to a remote location where the step of feeding is to occur. The method 10 includes applying a second beneficial substance 36 to the live food animal after the step of shipping.

The following are statements of exemplary embodiments of the invention:
1. A method of treating an animal, comprising:
   a. providing a live food creature configured to be consumable to an animal to be treated;
   b. applying a beneficial substance to the live food creature such that when consumed, the beneficial substance is consumed along with the live food creature;
   c. feeding the live food creature to the animal to be treated.
2. The method of Statement 1, wherein the beneficial substance is selected from the group of beneficial substances consisting of: vitamin supplements, color-enhancers, medicine, anti-fungal agents, antibiotics, probiotics, and antioxidants.
3. The method of Statement 1, wherein the live food creature is selected from the group of live food creatures consisting of worms, crickets, fish, and shrimp and wherein the animal to be treated is selected from the group of animals consisting of fish; frogs, reptiles, and spiders.
4. The method of Statement 1, wherein the step of applying consists essentially of injecting the beneficial substance into the live food creature.
5. The method of Statement 1, wherein the step of applying consists essentially of causing the live food creature to ingest the beneficial substance.
6. The method of Statement 1, wherein the step of applying consists essentially of causing the beneficial substance to be absorbed into the live food creature transdermally.
7. The method of Statement 1, further comprising the step of causing the live food creature to have a lower metabolic rate, thereby reducing the rate at which an effective dosage level of the beneficial substance is reduced.
8. The method of Statement 7, wherein the step of causing the live food creature to have a lower metabolic rate is selected from the group of rate lowering methods consisting of temperature reduction, light treatment, and chemically induced hibernation.
9. The method of Statement 1, further comprising the step of marking the live food creature according to a protocol configured to identify the live food creature as having a particular characteristic associated with the step of applying a beneficial substance to the live food creature.
10. The method of Statement 9, wherein the particular characteristic is selected from the group of characteristics consisting of beneficial substance name, application date, metabolic rate, shelf life, and dose level.
11. The method of Statement 1, further comprising the step of shipping the live food creature to a remote location where the step of feeding is to occur.
12. The method of Statement 9, wherein the step of marking is performed according to a marking technique selected from the group of marking techniques consisting of dying, tattooing, etching, and cutting.
13. The method of Statement 11, further comprising applying a second beneficial substance to the live food animal after the step of shipping.
14. A method of preparing a live food creature for beneficial feeding to an animal, comprising:
   a. providing a live food creature configured to be consumable to an animal to be treated;
   b. applying a beneficial substance to the live food creature such that when consumed, the beneficial substance is consumed along with the live food creature; and
   c. causing the live food creature to have a lower metabolic rate, thereby reducing the rate at which an effective dosage level of the beneficial substance is reduced.
15. The method of Statement 14, further comprising the step of marking the live food creature according to a protocol configured to identify the live food creature as having a particular characteristic associated with the step of applying a beneficial substance to the live food creature.
16. The method of Statement 15, wherein the particular characteristic is selected from the group of characteristics consisting of beneficial substance name, application date, metabolic rate, shelf life, and dose level.
17. The method of Statement 16, wherein the beneficial substance is selected from the group of beneficial substances consisting of: vitamin supplements, color-enhancers, medicine, anti-fungal agents, antibiotics, probiotics, and antioxidants.
18. The method of Statement 17, wherein the step of causing the live food creature to have a lower metabolic rate is selected from the group of rate lowering methods consisting of temperature reduction, light treatment, and chemically induced hibernation.
19. The method of Statement 18, further comprising applying a second beneficial substance to the live food animal after the step of shipping.
20. A live food creature comprising a beneficial substance not natural to the live food creature.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, although the figures illustrate injecting a worm with a foreign substance, it is understood that there are other ways to apply a foreign substance to a worm.

Additionally, although the figures illustrate fish and worms, it is understood that a great variety of animal/live food creature combinations may be used when practicing the invention.

It is also envisioned that the illustrated steps/methods/processes/etc. may be taken and/or may occur in orders other than that illustrated. Further, particular steps may occur more than once in a process. As a non-limiting example, a live food creature may have a foreign substance applied thereto and then may be fed to another live food creature, which is then fed to an animal to be treated. Advantageously, such a chained application of the steps described herein may provide substantial advantages to the process. For example, it may be that a particular foreign substance may be unpalatable to the primary live food creature associated with the animal to be treated and therefore insertion of an additional live food creature may permit an optimized treatment. As another non-limiting example, a foreign substance may be metabolized too quickly in a primary live food creature and insertion of an additional live food creature as hosting the foreign substance may beneficially extend the effective dose life of the system.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A method of treating an animal comprising the steps of:
a. providing a live food creature configured to be consumable to an animal to be treated;
b. applying a beneficial substance to the live food creature such that when consumed, the beneficial substance is consumed along with the live food creature;
c. feeding the live food creature to the animal to be treated.

2. The method of claim 1, wherein the beneficial substance is selected from the group of beneficial substances consisting of: vitamin supplements, color enhancers, medicine, anti-fungal agents, antibiotics, probiotics, and antioxidants.

3. The method of claim 1, wherein the live food creature is selected from the group of live food creatures consisting of worms, crickets, fish, and shrimp and wherein the animal to be treated is selected from the group of animals consisting of fish; frogs, reptiles, and spiders.

4. The method of claim 1, wherein the step of applying consists essentially of injecting the beneficial substance into the live food creature.

5. The method of claim 1, wherein the step of applying consists essentially of causing the live food creature to ingest the beneficial substance.

6. The method of claim 1, wherein the step of applying consists essentially of causing the beneficial substance to be absorbed into the live food creature transdermally.

7. The method of claim 1, further comprising the step of causing the live food creature to have a lower metabolic rate, thereby reducing the rate at which an effective dosage level of the beneficial substance is reduced.

8. The method of claim 7, wherein the step of causing the live food creature to have a lower metabolic rate is selected from the group of rate lowering methods consisting of temperature reduction, light treatment, and chemically induced hibernation.

9. The method of claim 1, further comprising the step of marking the live food creature according to a protocol configured to identify the live food creature as having a particular characteristic associated with the step of applying a beneficial substance to the live food creature.

10. The method of claim 9, wherein the particular characteristic is selected from the group of characteristics consisting of beneficial substance name, application date, metabolic rate, shelf life, and dose level.

11. The method of claim 1, further comprising the step of shipping the live food creature to a remote location where the step of feeding is to occur.

12. The method of claim 9, wherein the step of marking is performed according to a marking technique selected from the group of marking techniques consisting of dying, tattooing, etching, and cutting.

13. The method of claim 1, further comprising applying a second beneficial substance to the live food animal after the step of shipping.

14. A method of preparing a live food creature for beneficial feeding to an animal, comprising:
a. providing a live food creature configured to be consumable to an animal to be treated;
b. applying a beneficial substance to the live food creature such that when consumed, the beneficial substance is consumed along with the live food creature; and
c. causing the live food creature to have a lower metabolic rate, thereby reducing the rate at which an effective dosage level of the beneficial substance is reduced.

15. The method of claim 14, further comprising the step of marking the live food creature according to a protocol configured to identify the live food creature as having a particular characteristic associated with the step of applying a beneficial substance to the live food creature.

16. The method of claim 15, wherein the particular characteristic is selected from the group of characteristics consisting of beneficial substance name, application date, metabolic rate, shelf life, and dose level.

17. The method of claim 16, wherein the beneficial substance is selected from the group of beneficial substances consisting of: vitamin supplements, color-enhancers, medicine, anti-fungal agents, antibiotics, probiotics, and antioxidants.

18. The method of claim 17, wherein the step of causing the live food creature to have a lower metabolic rate is selected from the group of rate lowering methods consisting of temperature reduction, light treatment, and chemically induced hibernation.

19. The method of claim 18, further comprising applying a second beneficial substance to the live food animal after the step of shipping.

20. A live food creature comprising a beneficial substance not natural to the live food creature.
